(19) United States
(12) Patent Application Publication
(10) Pub. No.: US 2017/0367827 A1
(43) Pub. Date: Dec. 28, 2017

(54) MEDICAL IMPLANTS WITH 100% SUBSURFACE BORON CARBIDE DIFFUSION LAYER

(71) Applicant: Arthrex, Inc., Naples, FL (US)

(72) Inventors: Matthew Fonte, Concord, MA (US); Matthew Palmer, Medford, CA (US); Robert Devaney, Auburndale, MA (US)

(21) Appl. No.: 15/628,730

(22) Filed: Jun. 21, 2017

Related U.S. Application Data
(60) Provisional application No. 62/354,862, filed on Jun. 27, 2016.

(51) Int. Cl. A61F 2/30; A61F 2/38; B81C 1/00; A61L 27/04

(52) U.S. Cl. CPC A61F 2/30767; B81C 1/00444; A61F 2/38; A61F 2/30; A61F 2/3094; A61L 27/04

(57) ABSTRACT

An orthopedic medical implant, implant part or surgical instrument includes a metallic body having a metal or a metal alloy. The metallic body includes a sub-surface that is a thermal diffused boron carbide layer, and the metallic body is void of an additive layer onto a surface of the metallic body.

### CROSS REFERENCE TO RELATED APPLICATION

The application claims priority to U.S. Provisional Application No. 62/354,862 which was filed on Jun. 27, 2016, the entire disclosure of which is hereby incorporated by reference.

### FIELD OF THE INVENTION

The present invention provides metallic medical implants, medical implant parts, or surgical instruments having a surface at least partially including a fully diffused boronized metal layer to create a medical implant or medical implant part with enhanced surface hardness, increased lubricity, decreased coefficient of friction, and no risk of delamination. Further, the invention provides a process for producing a medical implant, medical implant part, or surgical instrument that at least partially includes a boronized metal diffused layer to create a medical implant, medical implant part, or surgical instrument with enhanced surface hardness, increased lubricity, decreased coefficient of friction, and no risk of delamination.

The present invention finds particular utility in the field of orthopedics, specifically for enhancing the surface properties of implants used for joint reconstruction. While the present invention has application throughout the body, its utility will be illustrated in the context of improving the surface properties of hip, knee, elbow, wrist, shoulder, ankle and vertebral implants.

### BACKGROUND OF THE INVENTION

In the field of orthopedic surgery, it is common to replace diseased or damaged joints. Orthopedic implants must endure significant mechanical stresses. Therefore, the materials from which the implants are made must combine high surface hardness, high lubricity, and a low coefficient of friction.

To date, the industry has looked to surface coatings to enhance the surface properties of implants and implant parts. United States Patent Application No. US2006/0074491 provides a medical implant or implant part that is coated with a boronized metal layer. U.S. Pat. No. 5,123,924 (entitled Surgical Implants and Method) discloses ion implantation of cobalt-chromium components to increase the microhardness of the implant and decrease the coefficient of friction. Both of these disclosures create an additive layer, thus changing the dimensions of the part.

Medical implants or medical implant parts include, but are not limited to, the femoral component of an uni-compartmental knee arthroplasty or a total knee arthroplasty, the tibial component of a uni-compartment knee arthroplasty or a total knee arthroplasty, the femoral head of hip arthroplasty, the Morse taper of a hip arthroplasty, the acetabular cup or liner of a hip arthroplasty, the humeral head of a shoulder arthroplasty, the humeral or ulnar component of an elbow arthroplasty, the metacarpal or radial stem of a wrist arthroplasty, the vertebral endplate components of a disc arthroplasty, and the tibial or talar component of an ankle arthroplasty.

One of the variables affecting the longevity of load-bearing implants is the rate of wear of the implant's articulating surface. Wearing of the articular surface generates debris particles that are released into the tissues surrounding the implant. Debris particles contribute to bone loss at the interface of the implant and the bone, contributing to osteolysis. The rate of wear of the articulating surface depends on the relative hardness, lubricity, and coefficient of friction of the materials from which the articulating surfaces are made.

Another variable that affects the longevity of load-bearing implants is the rate of wear at the connections between implant parts. These include, but are not limited to, the taper-femoral head interface of hip-joint implants and the interfaces of modular stems and necks of hip-joint implants. Taper fretting and crevice corrosion lead to the formation of debris particles, contributing to bone loss at the interface of the implant and bone as well as loss of implant mechanical strength. The rate of fretting and crevice corrosion of the interlocking connections depends on the relative hardness of the materials from which the articulating surfaces are made.

Orthopedic medical implants or medical implant parts are commonly manufactured from a metal or metal alloy selected from the group consisting of cobalt, cobalt alloys, titanium, titanium alloys, and mixtures thereof. Suitable cobalt-chromium alloys include, but are not limited to, the cast, forged, and wrought cobalt-28-chromium-6-molydenum (Co28Cr6Mo) alloys described in ASTM F75, F799, F90, F562 and F1537 or Cobalt alloy L605 (Haynes-Stellite 25) per ASTM F90-14 Standard Specification for Wrought cobalt-20-chromium-15-tungsten-10-nickel alloy for Surgical Implant Applications (UNS R30605). Suitable titanium alloys include, but are not limited to, the titanium-3-aluminum-2.5-vanadium alloys (Ti3Al2.5V) described in ASTM F2146, the titanium-6-aluminum-4-vanadium (Ti6Al4V) alloy described in ASTM F135, and the titanium-13-niobium-13-zirconium (Ti—13Nb—13Zr) alloy described in ASTM F1713.

Orthopedic implants have been developed which are made from relatively hard, wear-resistant oxide-ceramics. However, ceramic implants are often brittle and lack the toughness of metallic implants. Other implants have been made with a blue-black or black zirconium oxide coating. However, these coatings are very thin and have limited abrasion or scratch resistance. Thicker coatings result in delamination of the zirconium oxide layer from the base zirconium alloy substrate. Other efforts have been aimed at utilizing ion bombardment of the implant or coating the surface with diamond-like carbon (DLC) or titanium nitride. However, these efforts result in limited adhesion to the substrate and may be relatively thin and may not exhibit high peak hardness.

Thus, there exists a need for metallic orthopedic implants or implant parts with enhanced surface hardness, increased lubricity, and decreased coefficient of friction. A need also exits for a process for producing orthopedic implants or implant parts with enhanced surface hardness, increased lubricity, and decreased coefficient of friction.

### SUMMARY

The present invention provides a medical implant, an implant part or a surgical instrument including a fully diffused boronized subsurface of the metal or metal alloy. The boronized subsurface provides an extremely hard surface that is highly lubricious and has a very low coefficient of friction. The present invention also provides a method of producing a medical implant, implant part or surgical instrument including a diffused boronized subsurface of the metal or metal alloy.

The present invention includes a fully diffused surface treatment that is extremely hard and does not alter the implant or implant part's dimensions, while penetrating deeper into the implant or implant part's substrate. Because the present invention is diffused 100% into the implant's subsurface, there is not an additive coating that 1) can delaminate or wear off, 2) can change the implant's size/shape, or 3) will need to be ground and/or polished during manufacturing.

Among other things, the present invention also provides a diffusion surface treatment of the implant or implant part with a slurry containing boron carbide and other elements. A method includes heat treating the implant or implant part in an inert atmosphere and initiating the migration of boron atoms into the substrate. The migration process results in a surface chemistry high in boron.

The surface characteristic of orthopedic implants is critical in determining the durability of implants. The implant surface is directly exposed to attack from wear, fretting, and the corrosive body environment. Efforts to protect surfaces against these forms of attack are therefore commensurately great.

Thermochemical processes for increasing surface hardness have gained importance for the wear protection of ferrous metals, such as steels. Nitriding, nitrocarburizing, case-hardening, and boronizing are the thermochemical processes most frequently utilized for these ferrous metals.

Boronizing is a thermochemical diffusion process in which hard boride layers are generated by the diffusion of boron into the material surface.

During boronization, which is usually carried out at temperatures between 700° C. and 900° C., boron atoms diffuse into the material surface where they form very hard metal borides. The structure and properties of the boron layers are decisively determined by the base material. On ferrous materials, boride layers reach hardnesses of between 1800 and 2000 Vickers. On cobalt, chromium, and nickel based alloys, hardnesses may be as great as up to 2800 Vickers. Under contemporary boronization methods, depending on the particular application and base material, the layers are formed in thicknesses between 20 μm and 300 μm.

As with all surface treatment methods, boronizing has certain demands on the workpiece to be coated. For example, before the part is embedded in the boronizing agent, it must be washed to remove all adhering cutting fluids and corrosion protection oils.

Traditionally, boronizing increases the size of parts by approximately 20-30% of the overall boride layer thickness. In the case of parts that have to meet tight dimensional requirements, this effect is compensated for by appropriately undersizing the original workpiece during manufacturing. The application of the additive boron coating results in the final parts meeting dimensional requirement.

The additive layer of the post-boronized part may increase the surface roughness. Complex, multi-curved knee condyles, acetabular cups, and hip implant balls all require very smooth surface finishes and thus need to be ground and polished post-boronization. Post-boronization polishing requires expensive contour or profile grinding tools and often requires time consuming polishing with diamond hand pads for touch-ups. This causes boronized medical implants and implant parts to not be practical or manufacturable in production.

The present invention is different compared to traditional boronzing because the boron carbide layer is a fully diffused surface treatment that does not alter the dimensions of the implant or implant part. Unlike current boronizing surface treatments and boronized coatings that increase the dimensions of the implant or implant part, the present invention's boron carbide process does not alter the original dimensions of the implant or implant part. Traditional boronization processes result in surface growth that is generally 15% to 30% of the boron layer depth. The present invention is not a coating. There is not a positive layer to wear off and there is no surface coating. Rather, the hard boron carbide B₄C layer is diffused into the surface of the implant. The B₄C layer is 100% subsurface. This is a great advancement over other boronizing process that generate a boronized coating with roughly ⅔ its thickness diffused below the parts surface, and roughly ⅓ its thickness as an additive layer. Again, this additive layer often needs to be ground off, which is difficult and expensive to accomplish at such high hard hardness levels. This invention allows for implant surfaces to be finish machined, ground, and polished when still metallic (and thus still relatively soft), then treated to increase the surface hardness with the boron carbide diffusion process. The resulting parts do not need to be ground or polished. This is new to the industry.

The present invention also provides a surface treatment that is highly resistant to delamination. Existing surface treatments depend on the effective bond between the coating and the substrate to prevent delamination. Delamination of the coating from the substrate is a leading cause of coating failure. Because the boron carbide process of the current invention diffuses into the implant or implant part, the risk of delamination is eliminated.

In one form of the present invention, the implant or implant part has been boron carbonized without changing the implant's dimensions, has a surface hardness of 1,500 HK to 2,800 HK, has a surface chemistry of 40%-60% boron, and a coefficient of friction of 0.01 at 15,000 psi. The depth of boron diffusion is about 250 microns.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and wherein:

FIG. 1 illustrates a table showing the different cobalt alloys typically used in medical applications;

FIG. 2 illustrates a table showing how the addition of molybdenum, chromium, iron, nickel, and carbon affect the crystal structure of a cobalt;

FIG. 3 illustrates a table showing the weight percent of the hexagonal close packed phase resulting from different thermos-mechanical reduction of area for a cobalt-based alloy with 28% chromium and 6% molybdenum;

FIG. 4 illustrates a table showing the weight percent of the hexagonal close packed phase at varying reductions of cross-sectional area;

FIG. 5 illustrates a graph showing hardness for percent reduction of cross-sectional area for a cobalt-based alloy bar;

FIG. 6 illustrates a graph showing the aging response of a cobalt-based alloy; the graph shows the hardness versus aging time;

FIG. 7 illustrates a schematic view showing the components of a traditional hip arthroplasty;

FIG. 8 illustrates a schematic view showing the boronization process;

FIGS. 9 and 10 illustrate schematics showing the diffused boronized layer;

FIG. 11 illustrates a schematic showing the surface hardness of the boronized layer;

FIG. 12 illustrates a schematic showing a typical femoral head manufactured from a cobalt-based alloy;

FIG. 13 illustrates a schematic showing a modular component of a femoral arthroplasty;

FIG. 14 illustrates the energy dispersive spectroscopy (EDS) spectrum analysis of the B₄C layer through a scanning electronic microscope in cobalt alloy L605 per ASTM F90-14 Standard Specification for Wrought Cobalt-20-Chromium-15-Tungsten-10-Nickel Alloy for Surgical Implant Applications (UNS R30605);

FIGS. 15 and 16 illustrate scanning electronic microscope images of the B₄C coated sample processed at 1600° F. and 1750° F.;

FIG. 17 illustrates the thickness of the Co₂B layer and the hardness of the layer and the hardness of the core material; and

FIG. 18 illustrates an orthopedic cutting instrument having the Co₂B layer.

### DETAILED DESCRIPTION

Boronizing is a thermochemical diffusion process in which hard boride layers are generated by the diffusion of boron into the material surface. A medical implant, implant part or surgical instrument includes a fully diffused boronized subsurface of metal or metal alloy. The boronized subsurface provides an extremely hard surface that is highly lubricious and has a very low coefficient of friction. Additionally, a method of producing a medical implant, implant part or surgical instrument includes a diffused boronized subsurface of the metal or metal alloy.

A B₄C diffusion process provides a fully diffused surface treatment that is extremely hard and does not alter the implant or implant part's dimensions, while penetrating deeper into the implant or implant part's substrate. Because the layer is diffused 100% into the implant's subsurface, there is no additive coating that 1) can delaminate or wear off, 2) can change the implant's size/shape, or 3) will need to be ground and/or polished during manufacturing.

Among other things, the diffusion surface treatment of the implant or implant part provides a slurry containing boron carbide and other elements. A method includes heat treating the implant or implant part in an inert atmosphere and initiating the migration of boron atoms into the substrate. The migration process results in a surface chemistry high in boron.

The implant surfaces can be finish machined, ground, and polished when still metallic (and thus still relatively soft), and then treated to increase the surface hardness with the boron carbide diffusion process. The resulting parts do not need to be ground or polished.

A B₄C diffusion process transforms a surface of metal parts into an extremely hard and slick intermetallic non-brittle boride layer. The resulting diffused boride layer is not a coating. This eliminates the potential for bond failure that can result in delamination. Additionally, because the base material is transformed to an intermetallic boride from the original surface down to a desired penetration depth, zero dimensional change occurs through the process.

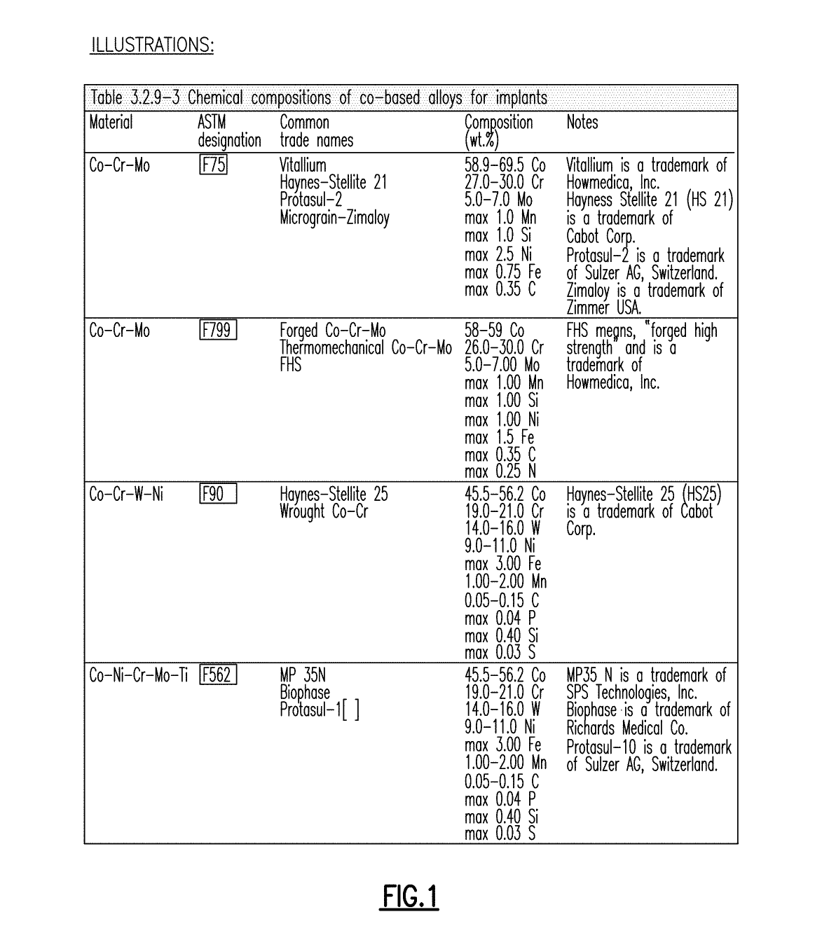

FIG. 1 illustrates a table defining several of the cobalt-based alloys used for medical applications. The cobalt-based alloys which are often used to produce orthopedic implants are well known and widely used in industry, primarily for wear applications. Many of the strength properties of the cobalt alloys arise from (1) the crystallographic texture of cobalt (Co), (2) the solid-solution-strengthening effects of Cr (chromium), W (tungsten) and Mo (molybdenum), (3) the formation of metal carbides, and (4) the corrosion resistance imparted by chromium. The cobalt alloy material has a high corrosion resistance, mainly due to the high chromium content that forms a thin passive chromium oxide layer with good adhesion, protecting the underlying matrix material.

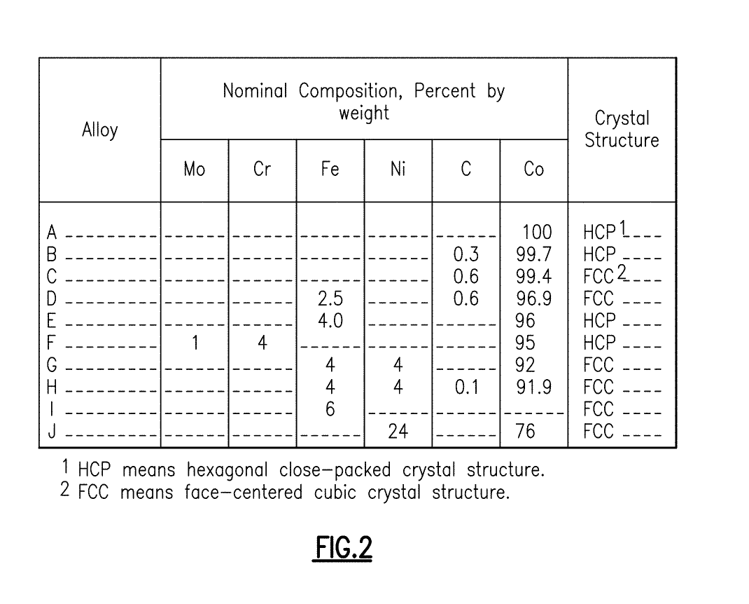

These alloys contain generally around 15% chromium to ensure a good corrosion resistance, between 4-17% tungsten or molybdenum for solid solution strengthening, and between 0.1-3% carbon to form hard carbides. The high temperature crystal structure of pure cobalt in its stable phase is face-centered cubic (fcc). Below 800° F., the crystal structure of the stable phase is hexagonal close packed (hcp). However, as shown in FIG. 2, the addition of carbon, iron and/or nickel to cobalt lowers the allotropic transformation point of the cobalt to a point below room temperature. The addition of these elements in effective amounts causes the face-centered cubic structure to exist at room temperature instead of the hexagonal close packed structure.

Additions of both chromium and tungsten tend to increase the transformation temperature in which the hexagonal close packed structure reverts back to face-centered cubic structure (at temperatures between 1769° F. and 2260° F., depending on the cobalt alloy).

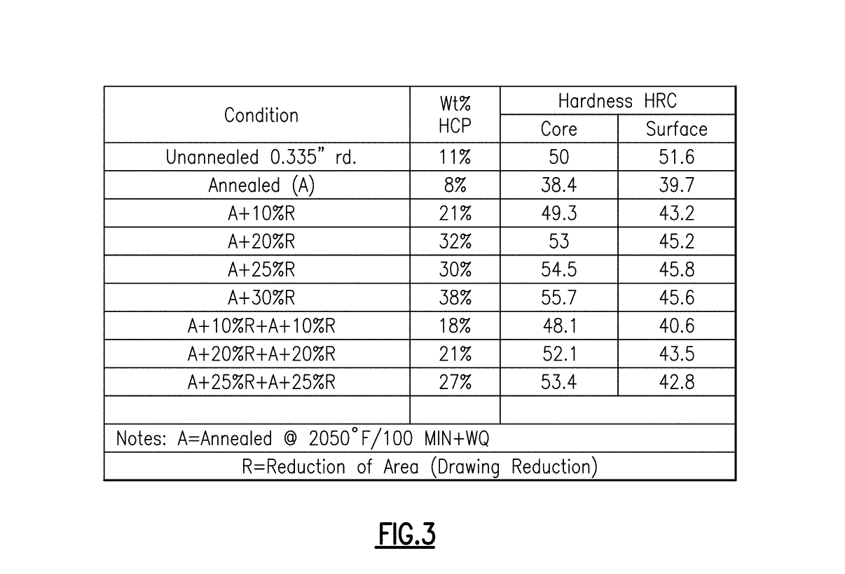

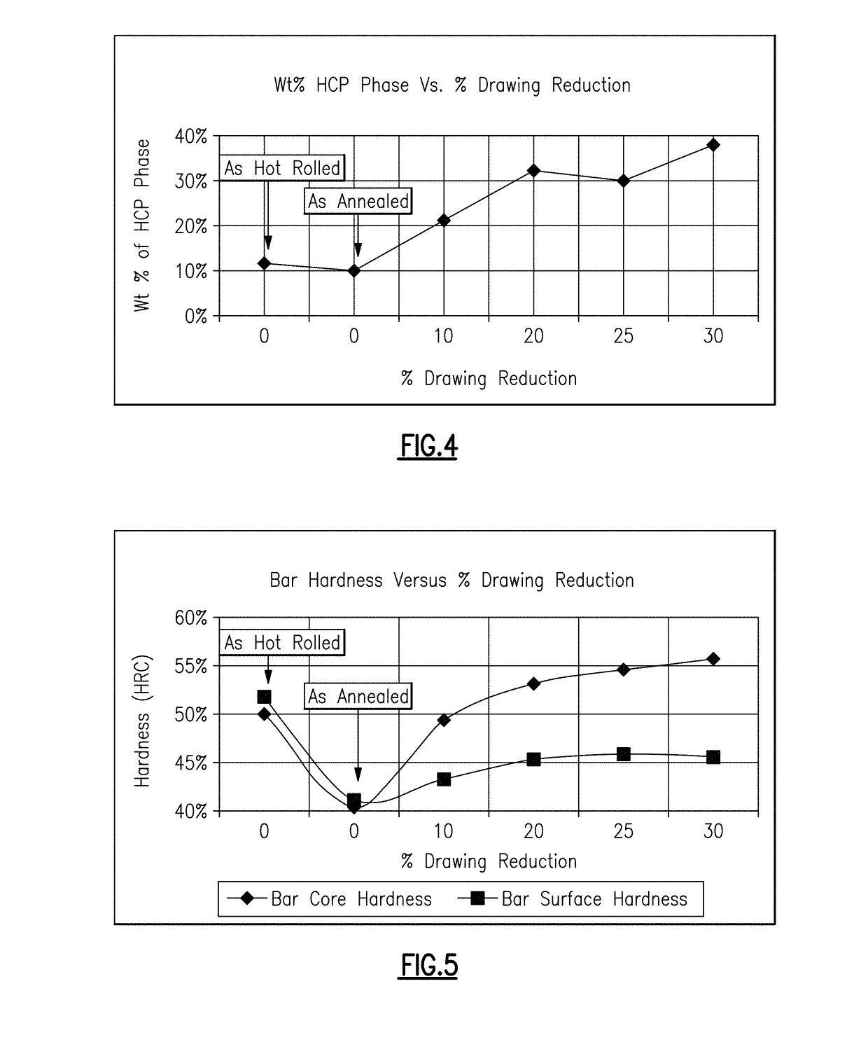

The face-centered cubic to hexagonal close packed crystallographic transformation occurs in cobalt alloys thru thermo-mechanical warm or cold working of the metal. Thermo-mechanical work is defined at processing/working (i.e. rolling, casting, forging, drawing, swaging) the metal at a temperature below its hexagonal close packed to face-centered cubic phase transformation temperature. However, the face-centered cubic crystal structure does not appear on the usual pressure-temperature phase diagrams of other hexagonal close packed alloys, such as titanium, zirconium, and hafnium (Hf), making this phenomenon unique to cobalt. For example, a cobalt-based alloy with 28% chromium and 6% molybdenum and having a 0.335″ diameter rod was cold drawn up to 30% in a single reduction operation. In this example, the microstructure underwent a 38% face-centered cubic to hexagonal close packed phase transformation. The percent of thermo-mechanical work reduction appears to increase the percent of face-centered cubic to hexagonal close packed phase transformation relatively linearly, such as shown in FIGS. 3 and 4.

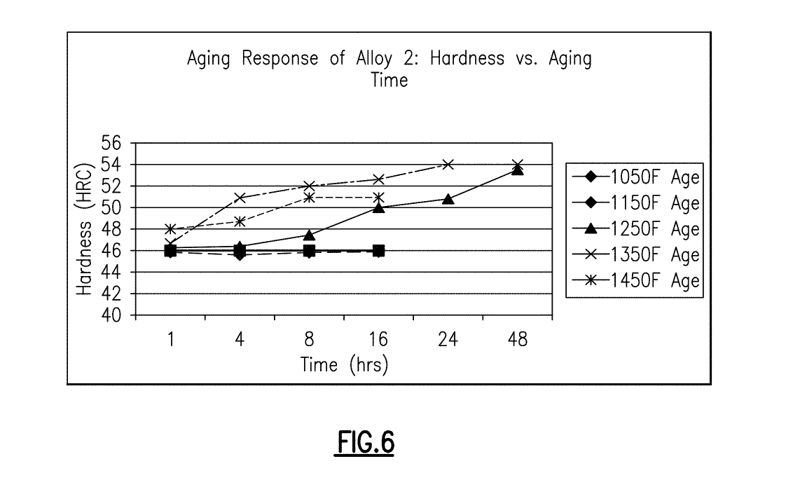

Because the cold or warm-drawing process (or forging) on a solid bar can only perform about a 30% reduction before needing an anneal operation, the reduced bar has 38% hexagonal close packed structure in its microstructure and the texturing effect is significant. The hexagonal close packed crystals are tightly locked together in the microstructure mixture (38% hexagonal close packed and 62% face-centered cubic), and the basal planes are radially oriented, increasing bi-axial strength and wear resistance (as shown in FIGS. 5 and 6).

The stresses built up from the thermo-mechanical (cold or warm) work of the cobalt alloys can only be relieved by annealing, which for most cobalt alloys is above 2,000° F. The anneal will reverse the hexagonal close packed to face-centered cubic phase transformation at temperatures between 1769° F. and 2260° F., depending on the cobalt alloy. At these temperatures, the desirable hexagonal close packed will revert to face-centered cubic and will stress relieve or anneal the material which is not desirable from a hardness and wear perspective.

Therefore, it is desirable to apply the B₄C thermal diffusion process to cobalt alloys at a temperature that is below the annealing temperature so not to soften the material and reverse the phase hexagonal close packed to face-centered cubic transformation, yet hot enough to allow for the boride layer to diffuse deep subsurface. Temperatures between 1000° F. and 1750° F. are appropriate for the B₄C diffusion process, will not reverse the favorable hexagonal close packed phase transformation, and will desirably age harden the implant.

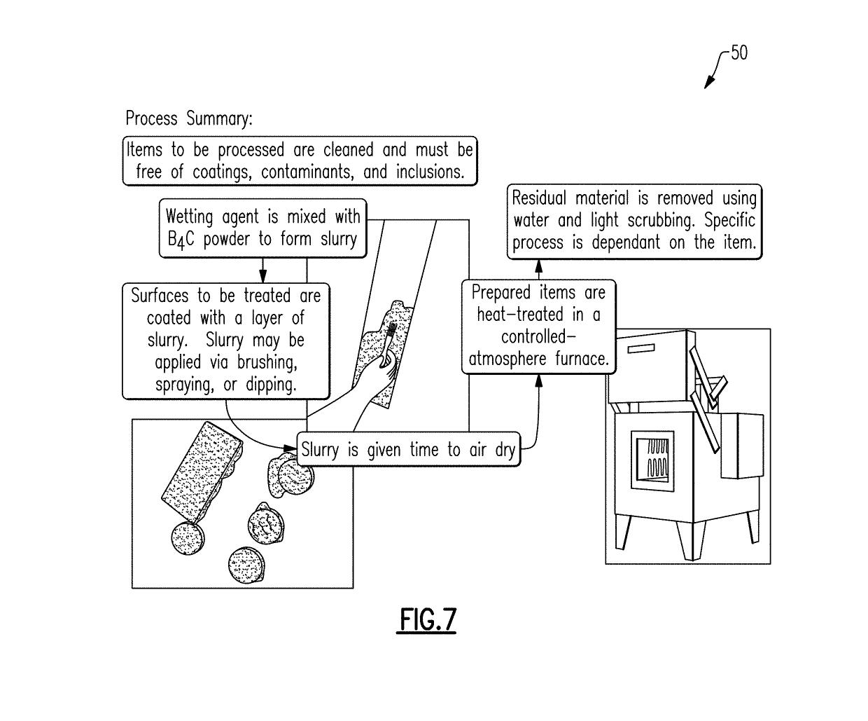

The present invention discloses a method for producing an implant or implant part that has a high surface hardness, is highly lubricious, has a low coefficient of friction, and resists delamination. In FIG. 7, the boron diffusion process 50 is shown. The process includes, cleaning the implant or implant part, creating a boron carbide slurry, coating the implant or implant part with the boron carbide slurry, allowing the slurry to dry, heat-treating the implant or implant part in a controlled atmosphere furnace, and removing residual material with water.

The heat-treatment may be achieved at a temperature of 1000° F. to 1700° F., with increasing temperatures resulting in increased depth of boron diffusion. The heat treatment may be performed under a protective-gas atmosphere such as argon, nitrogen, hydrogen, or a mixture of argon, nitrogen and hydrogen.

The B₄C slurry should be made using boron carbide nanopowder. The average particle size (APS) should be preferably between 45 nm to 55 nm and may be as small as 20 nm to 40 nm. This allows for 100% subsurface diffusion. The B₄C slurry may an activator, such as NaBF₄, KBF4, (NH₄)₃BF₄, NH₄Cl, Na₂CO₃, BaF₂, and Na₂B₃O₇. The slurry may also include a diluent, such as Al₂O₃.

It should be noted that if heat treatment at this temperature range results in the precipitation of carbides, the implant or implant part can be further processed to ensure that the ductility and toughness of the implant are not significantly negatively impacted. One method of doing this is by heating the implant or implant part to above 1200° C. to dissolve the carbides and then rapidly cooling the implant to about 800° C. to limit further carbide formation.

In one example, the boron carbide diffusion process occurs at below 1100° F., and the resulting medical implant or implant part is in the alpha+beta phase.

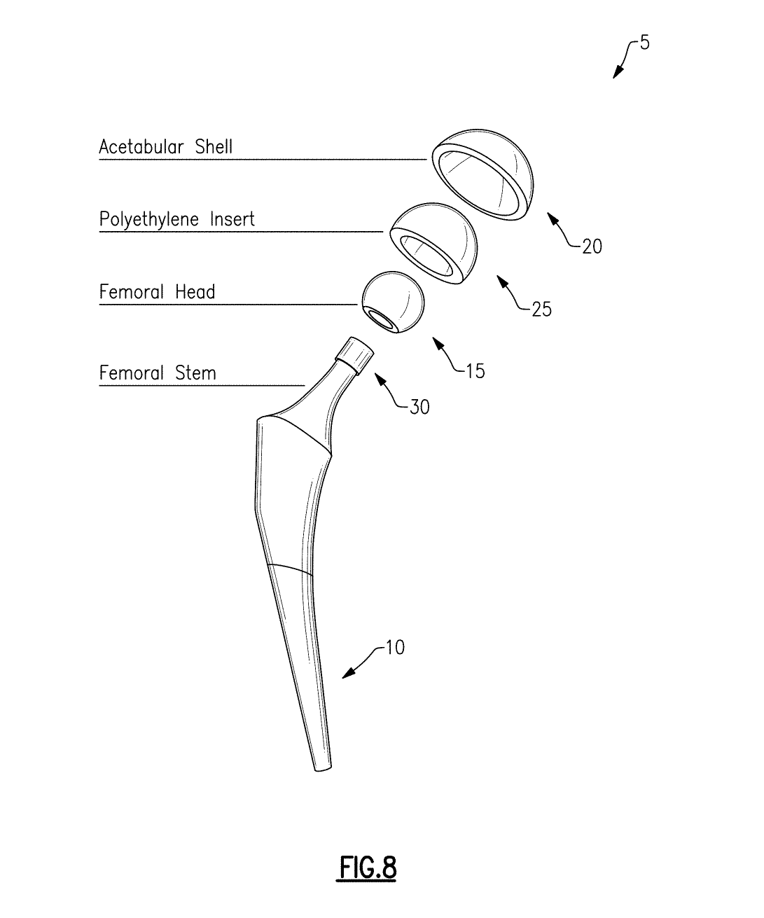

The boron diffusion process finds particular utility in increasing the surface performance of hip arthrodesis systems. FIG. 8 shows an exemplary hip arthrodesis system 5. The hip arthrodesis system consists of a stem 10, a femoral head 15, an acetabular cup 20, and an acetabular linear 25. The stem 10 is joined to the femoral head 15 by a taper 30. The femoral head 15 articulates inside the acetabular liner 25. Wear of the acetabular liner 25 can lead to failure of the hip arthrodesis system. It is highly desirable to have the femoral head 15 and the acetabular cup 20 have a high surface hardness, high lubricity, and a low coefficient of friction. It is further desirable to have a femoral head 15 that does not have an additive layer that can delaminate.

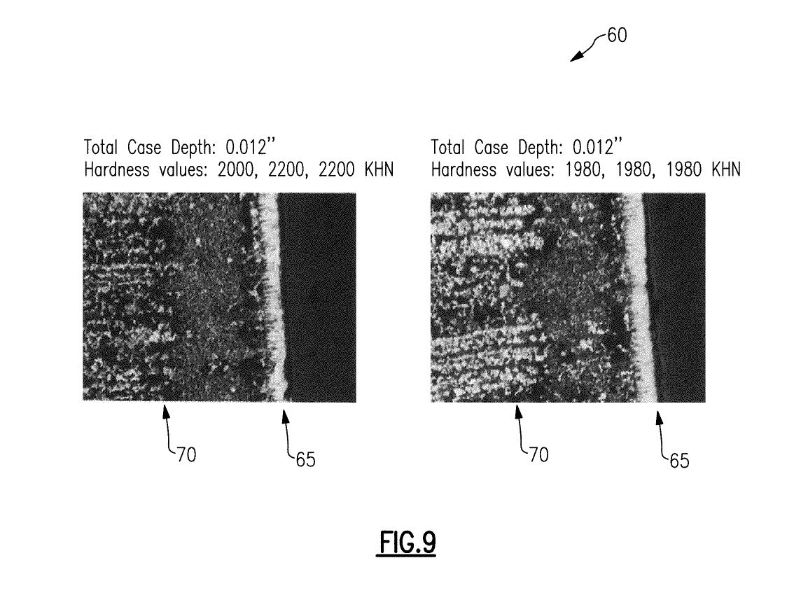

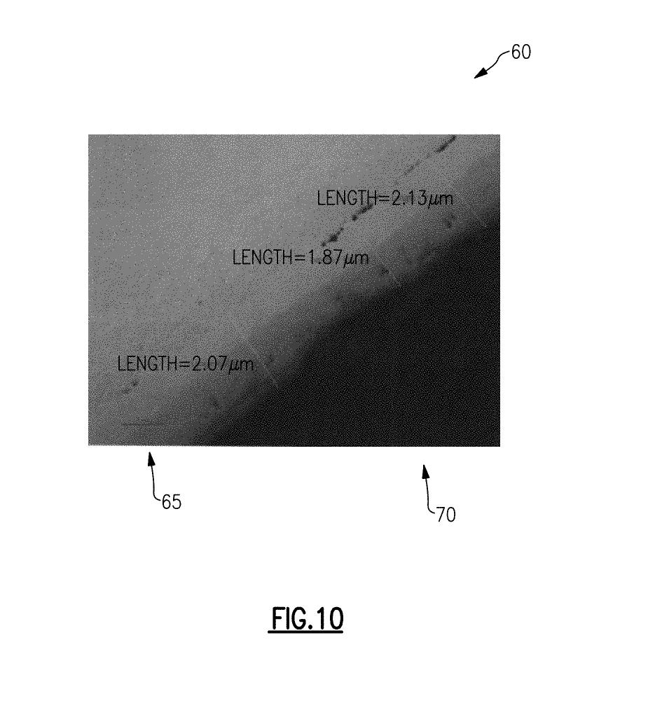

Now looking at FIGS. 9 and 10, the resulting implant or implant part is shown with an outer surface of diffused boron 60. The diffused boron layer 65 is shown adjacent to the substrate material 70. The diffused boron layer 65 may have a total depth of about 0.012″ (300 μm). It is important to note that the diffused boron coating does not change the dimensions of the implant or implant part.

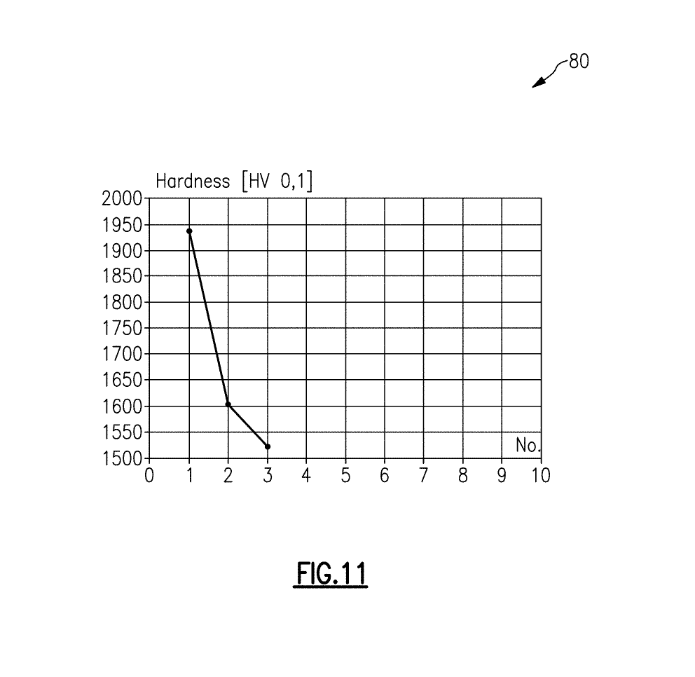

The boron coating is very hard. Looking now at FIG. 11, the surface hardness 80 may be between 1500 HV and 2000 HV.

### EXAMPLE 1

FIG. 12 shows a femoral head 90 that has been boronized using the B₄C diffusion process previously described. The femoral head 90 has a surface hardness of 1800 HK, a surface chemistry of 40%-60% boron, and a coefficient of friction of 0.01 at 15,000 psi. The depth of boron diffusion is about 250 microns.

### EXAMPLE 2

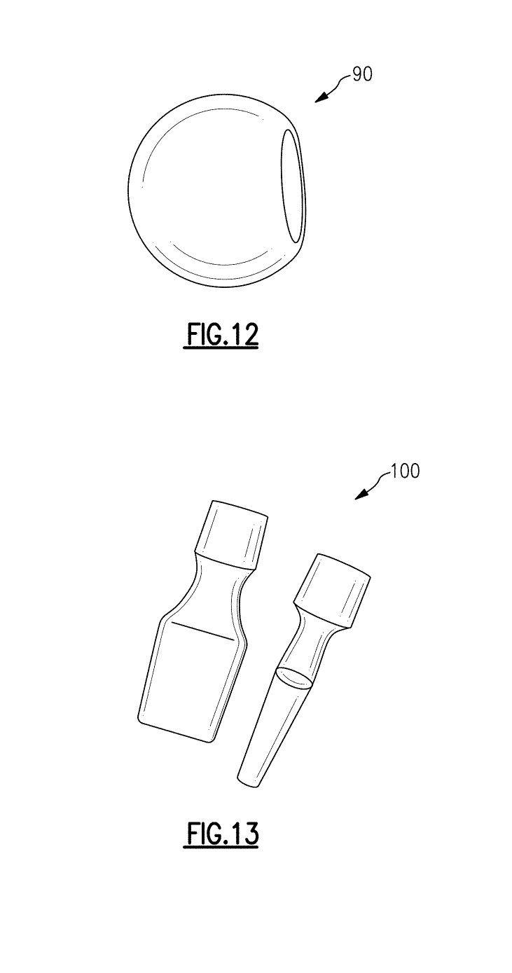

FIG. 13 shows the Morse taper region of a hip arthroplasty 100 that has been boronized using the process previously described. The more taper has a surface hardness of 1600 HK, a surface chemistry of 40%-60% boron, and a coefficient of friction of 0.01 at 15,000 psi. The depth of boron diffusion is about 150 microns.

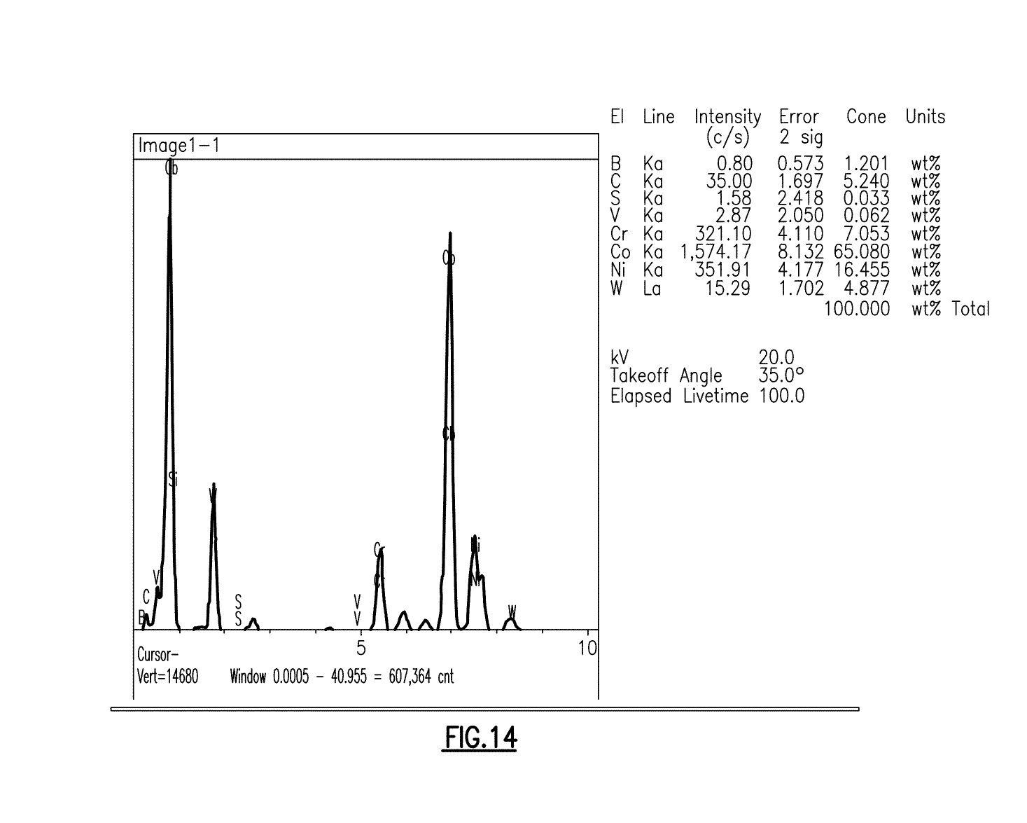

FIG. 14 shows the EDS (energy dispersive spectroscopy) spectrum analysis of the B₄C layer (shown through a scanning electronic microscope) in cobalt alloy L605 per ASTM F90—14 Standard Specification for Wrought Cobalt-20-Chromium-15-Tungsten-10-Nickel Alloy for Surgical Implant Applications (UNS R30605). An advantage to applying the B₄C diffusion level to alloy L605 is that the boronizing temperature up to 1700° F. is not hot enough to anneal the material, so the metal alloy can remain hard in its cold, worked, and aged condition with a hexagonal close packed crystallographic texture while its surface is the diffusion layer can be Co₃B or Co₂B.

### EXAMPLES 3 & 4

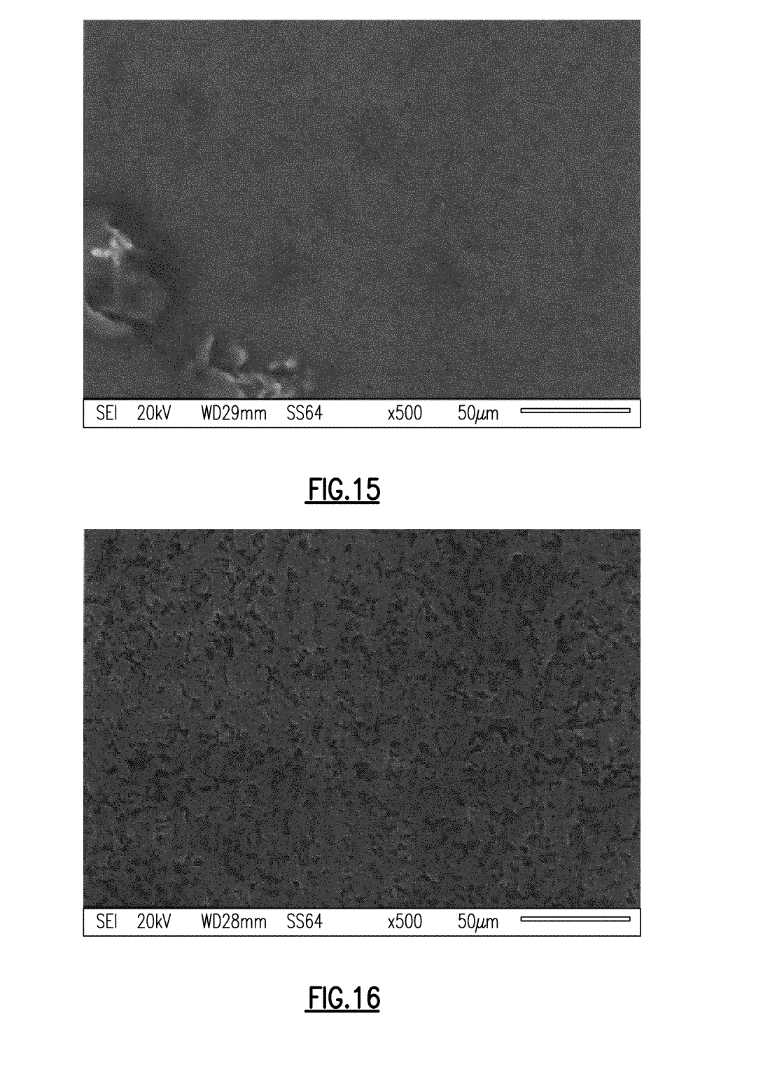

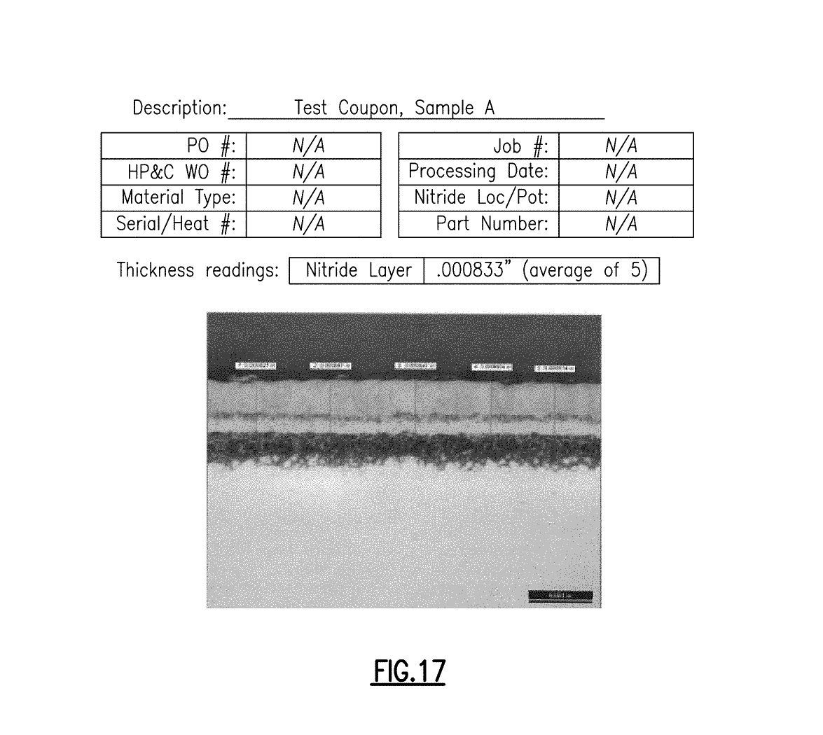

One B₄C diffusion sample was processed at 1600° F., and the other sample was processed at 1750° F. The 1600° F. sample has an average surface roughness of 6.5 μin, and the 1750° F. sample has an average surface roughness of 14.4 μin. The difference in surface roughness can be seen in FIGS. 15 and 16. FIG. 15 shows a scanning electronic microscope image of the B₄C diffusion process sample processed at 1600° F. The image shows a fairly smooth surface finish. FIG. 16 shows a scanning electronic microscope image of the B₄C thermal diffused sample processed at 1750° F. FIG. 16 shows a slightly rougher surface finish.

Looking now at FIG. 17, the Co₂B layer thickness was measured after B₄C diffusion into medical grade Co—Cr alloy. The average layer thickness was found to be 0.00083″. Knoop hardness measurements were also taken of the Co₂B layer and the core material. Knoop 25 g hardness values for the Co₂B surface were found to be on average 1929 (>80 HRC). The core material was found to have a Knoop 25 g hardness of 477 (46 HRC).

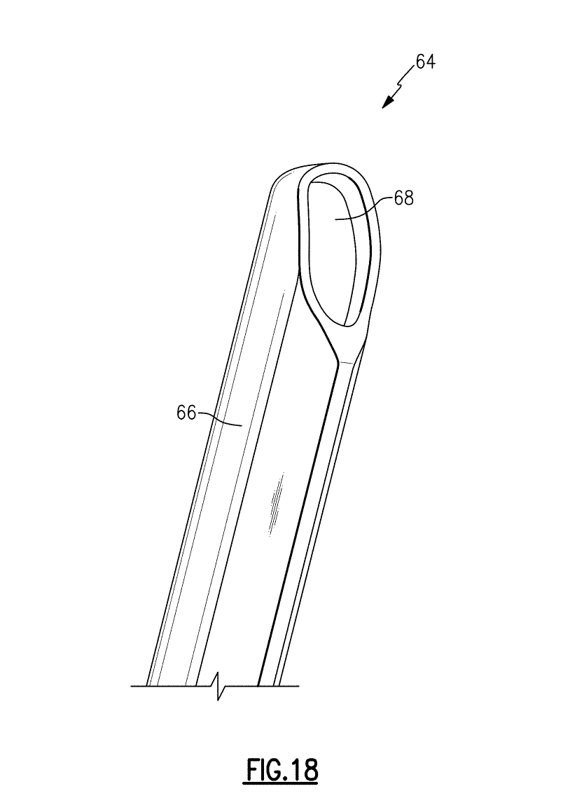

FIG. 18 shows an orthopedic cutting instrument 64. In one example, the orthopedic cutting instrument 64 is a resector or shaver that cuts cartilage and bone during surgery. The orthopedic cutting instrument 64 includes an outer shell 66 and a blade 68 that rotates in the outer shell 66 to remove cartilage and bone. In one example, the blade 68 rotates at 8000 RPMs.

During operation, the inner blade 68 can hit the outer shell 66 as the blade 68 rotates. In one example, the outer shell 66 is made of a cobalt-chromium alloy, and an inner surface of the outer shell 66 has a B₄C layer as described above. In this example, the blade 68 is made of a softer material than the inner surface of the outer shell 66. If the blade 68 and the outer shell 66 engage each other during operation, the softer blade 68 can wear, preventing breakage and any metal from entering the surgical site. The B₄C layer does not increase any dimension of the orthopedic cutting instrument 64, allowing for a tight fit between the rotating blade 68 and the outer shell 66. In another example, the blade 68 is made of a cobalt-chromium alloy with a B₄C layer, and the outer shell 66 is made of a softer material. In another example, both the outer shell 66 and the blade 68 are made of a cobalt-chromium alloy with a B₄C layer, but they have different hardnesses.

In one example, an orthopedic medical implant, implant part or surgical instrument includes a metallic body including a metal or a metal alloy. The metal or metal alloy is selected from the group consisting of cobalt, cobalt-chromium alloys, titanium, titanium-alloys, and mixtures thereof. The metallic body includes a sub-surface that is a thermal diffused boron carbide layer. The thermal diffused boron carbide layer comprises borides having a formula MeB, MeB², or Me₂B, where Me represents a metal present in the metallic body of the orthopedic medical implant, implant part or surgical instrument. The metallic body is void of an additive layer onto a surface of the metallic body, and a dimension of a pre-boronized implant or implant part is the same as a dimension of a post-boronized implant or implant part.

In another example, an orthopedic medical implant, implant part or surgical instrument includes a metallic body including a metal or a metal alloy. The metallic body includes a sub-surface that is a thermal diffused boron carbide layer, and the metallic body is void of an additive layer onto a surface of the metallic body.

In another embodiment according to any of the previous embodiments, the metal or metal alloy is selected from the group consisting of cobalt, cobalt-chromium alloys, titanium, titanium-alloys, and mixtures thereof.

In another embodiment according to any of the previous embodiments, the thermal diffused boron carbide layer includes borides having a formula MeB, MeB², or Me₂B, where Me represents a metal present in the metallic body of the orthopedic medical implant, implant part or surgical instrument.

In another embodiment according to any of the previous embodiments, a dimension of a pre-boronized implant or implant part is the same as a dimension of a post-boronized implant or implant part.

In another embodiment according to any of the previous embodiments, a surface hardness of the surface of the metallic body is at least 1500 HV.

In another embodiment according to any of the previous embodiments, a boronization thickness of the metallic body is at least 100 microns.

In another embodiment according to any of the previous embodiments, the surface of the metallic body has a coefficient of friction of 0.01 at 15,000 psi.

In another embodiment according to any of the previous embodiments, a surface chemistry of the surface of the metallic body is 40% to 60% boron.

In another embodiment according to any of the previous embodiments, the implant or implant part is selected from the group consisting of a femoral component of an uni-compartmental knee arthroplasty or a total knee arthroplasty, a tibial component of a uni-compartment knee arthroplasty or a total knee arthroplasty, a femoral head of hip arthroplasty, a Morse taper of a hip arthroplasty, an acetabular cup or liner of a hip arthroplasty, a humeral head of a shoulder arthroplasty, a humeral or ulnar component of an elbow arthroplasty, a metacarpal or radial stem of a wrist arthroplasty, a vertebral endplate components of a disc arthroplasty, and a tibial or talar component of an ankle arthroplasty.

In another embodiment according to any of the previous embodiments, the metallic body is a cobalt-based alloy.

In another embodiment according to any of the previous embodiments, the medical implant, implant part or surgical instrument has a hexagonal close packed crystal structure and is age hardened.

In another embodiment according to any of the previous embodiments, the metallic body is a titanium-based alloy.

In another embodiment according to any of the previous embodiments, orthopedic medical implant, implant part or surgical instrument is in an alpha+beta phase.

In another example, an orthopedic medical implant, implant part or surgical instrument includes a metallic body including a metal or metal alloy. The metallic body includes a sub-surface including a boronized layer of the metal or metal alloy. The orthopedic medical implant, implant part or surgical instrument is in an unannealed condition.

In another example, a method of forming an orthopedic medical implant, implant part or surgical instrument, the method includes creating a thermal diffused boron carbide layer in a sub-surface of a metallic body of the orthopedic medical implant, implant part or surgical instrument. The orthopedic medical implant, implant part or surgical instrument includes a metal or a metal alloy, and the metallic body is void of an additive layer onto a surface of the metallic body.

In another embodiment according to any of the previous embodiments, the method includes heat-treating the orthopedic medical implant, implant part or surgical instrument in a controlled atmosphere furnace.

In another embodiment according to any of the previous embodiments, the heating-treating occurs between 1000° F. and 1700° F.

In another embodiment according to any of the previous embodiments, the heating-treating occurs between 1000° F. and 1750° F., and the orthopedic medical implant, implant part or surgical instrument has a hexagonal close packed crystal structure and is age hardened.

In another embodiment according to any of the previous embodiments, the heating-treating occurs at below 1100° F., and the orthopedic medical implant, implant part or surgical instrument is in an alpha+beta phase.

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. An orthopedic medical implant, implant part or surgical instrument comprising:
a metallic body comprising a metal or a metal alloy, wherein the metal or metal alloy is selected from the group consisting of cobalt, cobalt-chromium alloys, titanium, titanium-alloys, and mixtures thereof, the metallic body includes a sub-surface that is a thermal diffused boron carbide layer, the thermal diffused boron carbide layer comprises borides having a formula MeB, MeB², or Me₂B, where Me represents a metal present in the metallic body of the orthopedic medical implant, implant part or surgical instrument, the metallic body is void of an additive layer onto a surface of the metallic body, and a dimension of a pre-boronized implant, implant part, or surgical instrument is the same as a dimension of a post-boronized implant, implant part, or surgical instrument.

2. An orthopedic medical implant, implant part or surgical instrument comprising:
a metallic body comprising a metal or a metal alloy, the metallic body including a sub-surface that is a thermal diffused boron carbide layer, and the metallic body is void of an additive layer onto a surface of the metallic body.

3. The orthopedic medical implant, implant part or surgical instrument as recited in claim 2 wherein the metal or metal alloy is selected from the group consisting of cobalt, cobalt-chromium alloys, titanium, titanium-alloys, and mixtures thereof.

4. The orthopedic medical implant, implant part or surgical instrument as recited in claim 2 wherein the thermal diffused boron carbide layer comprises borides having a formula MeB, MeB², or Me₂B, where Me represents a metal present in the metallic body of the orthopedic medical implant, implant part or surgical instrument.

5. The orthopedic medical implant, implant part or surgical instrument as recited in claim 2 wherein a dimension of a pre-boronized implant or implant part is the same as a dimension of a post-boronized implant or implant part.

6. The orthopedic medical implant, implant part or surgical instrument as recited in claim 2 wherein a surface hardness of the surface of the metallic body is at least 1500 HV.

7. The orthopedic medical implant, implant part or surgical instrument as recited in claim 2 wherein a boronization thickness of the metallic body is at least 100 microns.

8. The orthopedic medical implant, implant part or surgical instrument as recited in claim 2 wherein the surface of the metallic body has a coefficient of friction of 0.01 at 15,000 psi.

9. The orthopedic medical implant, implant part or surgical instrument as recited in claim 2 wherein a surface chemistry of the surface of the metallic body is 40% to 60% boron.

10. The orthopedic medical implant, implant part or surgical instrument as recited in claim 2 wherein the implant or implant part is selected from the group consisting of a femoral component of an uni-compartmental knee arthroplasty or a total knee arthroplasty, a tibial component of a uni-compartment knee arthroplasty or a total knee arthroplasty, a femoral head of hip arthroplasty, a Morse taper of a hip arthroplasty, an acetabular cup or liner of a hip arthroplasty, a humeral head of a shoulder arthroplasty, a humeral or ulnar component of an elbow arthroplasty, a metacarpal or radial stem of a wrist arthroplasty, a vertebral endplate components of a disc arthroplasty, and a tibial or talar component of an ankle arthroplasty.

11. The orthopedic medical implant, implant part or surgical instrument as recited in claim 2 wherein the metallic body is a cobalt-based alloy.

12. The orthopedic medical implant, implant part or surgical instrument as recited in claim 2 wherein the medical implant, implant part or surgical instrument has a hexagonal close packed crystal structure and is age hardened.

13. The orthopedic medical implant, implant part or surgical instrument as recited in claim 2 wherein the metallic body is a titanium-based alloy.

14. The orthopedic medical implant, implant part or surgical instrument as recited in claim 2 wherein orthopedic medical implant, implant part or surgical instrument is in an alpha+beta phase.

15. An orthopedic medical implant, implant part or surgical instrument comprising:
a metallic body comprising a metal or metal alloy, the metallic body including a sub-surface including a boronized layer of the metal or metal alloy, wherein the orthopedic medical implant, implant part or surgical instrument is in an unannealed condition.

16. A method of forming an orthopedic medical implant, implant part or surgical instrument, the method comprising creating a thermal diffused boron carbide layer in a sub-surface of a metallic body of the orthopedic medical implant, implant part or surgical instrument, wherein the orthopedic medical implant, implant part or surgical instrument comprises a metal or a metal alloy, and the metallic body is void of an additive layer onto a surface of the metallic body.

17. The method as recited in claim 16 further comprising heat-treating the orthopedic medical implant, implant part or surgical instrument in a controlled atmosphere furnace.

18. The method as recited in claim 17 wherein heating-treating occurs between 1000° F. and 1700° F.

19. The method as recited in claim 17 wherein heating-treating occurs between 1000° F. and 1750° F., and the orthopedic medical implant, implant part or surgical instrument has a hexagonal close packed crystal structure and is age hardened.

20. The method as recited in claim 17 wherein heating-treating occurs at below 1100° F., and the orthopedic medical implant, implant part or surgical instrument is in an alpha+beta phase.
